(19) United States
(12) Patent Application Publication
(10) Pub. No.: US 2024/0170115 A1
(43) Pub. Date: May 23, 2024

(54) AUTOMATED IDENTIFICATION OF PATIENT DISEASE CONTEXT USING EVIDENTIARY TIMELINES

(71) Applicant: KONINKLIJKE PHILIPS N.V., EINDHOVEN (NL)

(72) Inventors: Vadiraj HOMBAL, WAKEFIELD, MA (US); Thusitha Dananjaya De Silva MABOTUWANA, REDMOND, WA (US)

(21) Appl. No.: 18/279,606

(22) PCT Filed: Mar. 5, 2022
(86) PCT No.: PCT/EP2022/055655

§ 371 Date: Aug. 31, 2023

Related U.S. Application Data
(60) Provisional application No. 63/157,080, filed on Mar. 5, 2021.

(51) Int. Cl. G16H 15/00

(52) U.S. Cl. CPC G16H 15/00

(57) ABSTRACT

A method for classifying medical reports of a patient, including: receiving a plurality of patient medical reports; processing the plurality of patient medical reports to produce a processed report that extracts patient medical information; estimating the similarity between the plurality of medical reports based upon the extracted patient medical information; clustering similar medical reports; inferring a group type for the clustered medical reports and labeling the clustered medical reports with the inferred group type; and visualizing the labelled clustered medical reports on a display.

### TECHNICAL FIELD

Various exemplary embodiments disclosed herein relate generally to a system and method for automated identification of evidentiary timelines.

### BACKGROUND

In several clinical workflows, clinicians gather and analyze relevant patient records. Information in relevant prior radiology exams and lab results (including pathology exams) enables the clinicians to consider the patient disease context before arriving at the correct diagnosis, in deciding the appropriate treatment plan, or in evaluating the quality of their diagnoses.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method for classifying medical reports of a patient, including: receiving a plurality of patient medical reports; processing the plurality of patient medical reports to produce a processed report that extracts patient medical information; estimating the similarity between the plurality of medical reports based upon the extracted patient medical information; clustering similar medical reports; inferring a group type for the clustered medical reports and labeling the clustered medical reports with the inferred group type; and visualizing the labelled clustered medical reports on a display.

Various embodiments are described, wherein processing the plurality of patient medical reports includes document structure processing.

Various embodiments are described, wherein processing the plurality of patient medical reports includes syntactic parsing of an output of the document structure processing.

Various embodiments are described, wherein processing the plurality of patient medical reports includes extracting entities from an output of the syntactic parsing.

Various embodiments are described, wherein processing the plurality of patient medical reports includes determining an anatomy inference on the extracted entities.

Various embodiments are described, further including: receiving a current medical report for the patient; processing the current medical report to produce a current processed report that extracts patient medical information; estimating the similarity between the current medical report and the plurality of medical reports; determining which of the clusters of medical reports the current report is similar to; labeling the current medical report with the inferred group type associated with the determined cluster of medical reports; and visualizing the current medical report on a display.

Various embodiments are described, wherein the plurality of medical reports includes radiology reports and pathology reports.

Various embodiments are described, wherein clustering similar medical reports includes using a machine learning model.

Various embodiments are described, wherein clustering similar medical reports includes identifying medical reports that have a similarity score above a threshold value.

Various embodiments are described, further including determining a current anatomy label for the current report based upon the processing of the current medical report wherein clustering similar medical reports includes using current anatomy label to match anatomy labels on the plurality of medical reports produced by the report processing.

Various embodiments are described, wherein estimating the similarity between the plurality of medical reports is based upon one of anatomies identified in the reports, location of the disease identified in the reports, and the type of disease identified in the reports.

Further various embodiments relate to a device for classifying medical reports of a patient, including: a memory; a processor coupled to the memory, wherein the processor is further configured to: receive a plurality of patient medical reports; process the plurality of patient medical reports to produce a processed report that extracts patient medical information; estimate the similarity between the plurality of medical reports based upon the extracted patient medical information; cluster similar medical reports; infer a group type for the clustered medical reports and labeling the clustered medical reports with the inferred group type; and visualize the labelled clustered medical reports on a display.

Various embodiments are described, wherein processing the plurality of patient medical reports includes document structure processing.

Various embodiments are described, wherein processing the plurality of patient medical reports includes syntactic parsing of an output of the document structure processing.

Various embodiments are described, wherein processing the plurality of patient medical reports includes extracting entities from an output of the syntactic parsing.

Various embodiments are described, wherein processing the plurality of patient medical reports includes determining an anatomy inference on the extracted entities.

Various embodiments are described, wherein the processor is further configured to: receive a current medical report for the patient; process the current medical report to produce a current processed report that extracts patient medical information; estimate the similarity between the current medical report and the plurality of medical reports; determine which of the clusters of medical reports the current report is similar to; label the current medical report with the inferred group type associated with the determined cluster of medical reports; and visualize the current medical report on a display.

Various embodiments are described, wherein the plurality of medical reports include radiology reports and pathology reports.

Various embodiments are described, wherein clustering similar medical reports includes using a machine learning model.

Various embodiments are described, wherein clustering similar medical reports includes identifying medical reports that have a similarity score above a threshold value.

Various embodiments are described, wherein the processor is further configured to determine a current anatomy label for the current report based upon the processing of the current medical report wherein clustering similar medical reports includes using current anatomy label to match anatomy labels on the plurality of medical reports produced by the report processing.

Various embodiments are described, wherein estimating the similarity between the plurality of medical reports is based upon one of anatomies identified in the reports, location of the disease identified in the reports, and the type of disease identified in the reports.

### BRIEF DESCRIPTION OF THE DRAWINGS

In order to better understand various exemplary embodiments, reference is made to the accompanying drawings, wherein:

FIG. 1 illustrates an embodiment of a clustering system that groups patient radiology and pathology exams;

FIG. 2 illustrates a report processing pipeline of the patient reports carried out by the report processor and the inference processor;

FIG. 3 illustrates a flowchart of a method performed by a clustering system that groups medical exams; and

FIG. 4 illustrates an exemplary hardware device for implementing the clustering system.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

Patients may have an evidentiary timeline that includes various different information such as medical images, medical tests, medical examinations, etc. Further, patients (especially older patients) may have multiple morbidities. For example, a patient may have had a broken hip or other broken bones. Further, the patient may have had have other issues with the breast, liver, or other anatomies. Such issues may for example result in a large number of X-rays, MRIs, CT scans, or other medical images. For example, a patient may have 50 or more images in their medical records.

When the patient is then screened for cancer, (e.g., breast cancer, lung cancer, etc.) additional images are taken. If the prior images are grouped together, an examining physician can more effectively isolate relevant medical images for evaluation by comparing current images to the prior images. For example, the prior images may be grouped based upon musculoskeletal episodes, breast cancer, liver studies, lung studies, etc.

Patients often have complex clinical histories with multiple radiology and lab exams, and with multiple chronic pathologies. For example, a female patient may have a hip fracture, breast cancer, and incidental lung nodules. It is not uncommon for clinicians to consider dozens of exams to arrive at a correct relevant context. Gathering of relevant patient context in several clinical settings is performed manually and involves combinatorial complexity due to multiple pathologies. This may result in over-specification of the patient context, where all exams in the field of view are pulled, or result in under-specification, where exams not in the field of view but that are still relevant to patient condition are ignored.

In many radiology reading workflows, prior medical images are presented chronologically based on simple heuristics on exam digital imaging and communications in medicine (DICOM) data to gather relevant prior exams. These algorithms do not consider the semantic content of reports or the complexity of relationships between exams. Radiologists typically scroll through prior exams manually and select prior exams for review based on field of view (CT/MRI/X-ray Chest vs. CT/MRI/X-ray Pelvis). This manual selection process can lead to missing important relevant prior exams thus increasing the risk of misdiagnosis due to incomplete context. For example, in a metastatic disease, relevant prior exams might not be the same modality as current exams used in a diagnosis (e.g., X-ray mandible could be a relevant prior to MRI prostate).

Embodiments of a method and system will be described that may identify related radiology and pathology reports based on a parsing of the exams and identifying complex relationships between them. These embodiments provide a systematic solution for identifying complex relationships between exams.

The embodiments described herein provide a solution to classify patient radiology and pathology exams according to anatomy of disease/pathology enabling gathering of relevant patient data and presentation in different disease contexts. As an example, the focus is on identification of contexts relevant for the following clinical workflows (but the embodiments may be applied to other workflows as well).For a Radiology Pathology Correlation workflow when given a pathology report for a surgical procedure (e.g., biopsy, excision, etc.), an embodiment may identify relevant prior radiology and pathology exams for radiology and pathology outcome concordance. This application is fundamental to radiology clinical quality and training.

For a Radiology Reading Workflow when given a region of interest in the current radiology image being read, an embodiment may identify relevant prior radiology and pathology exams to obtain a patient exam context to understand disease onset, progression or spread, and treatment effectiveness. This application supports critical functions of radiologists.

For a Longitudinal Analyses to Identify Discrepant Reads workflow, an embodiment identifies discrepant reads for second-read or peer-review based on analysis of subsequent and prior exams. This application is critical to identification of mis-reads and clinical quality.

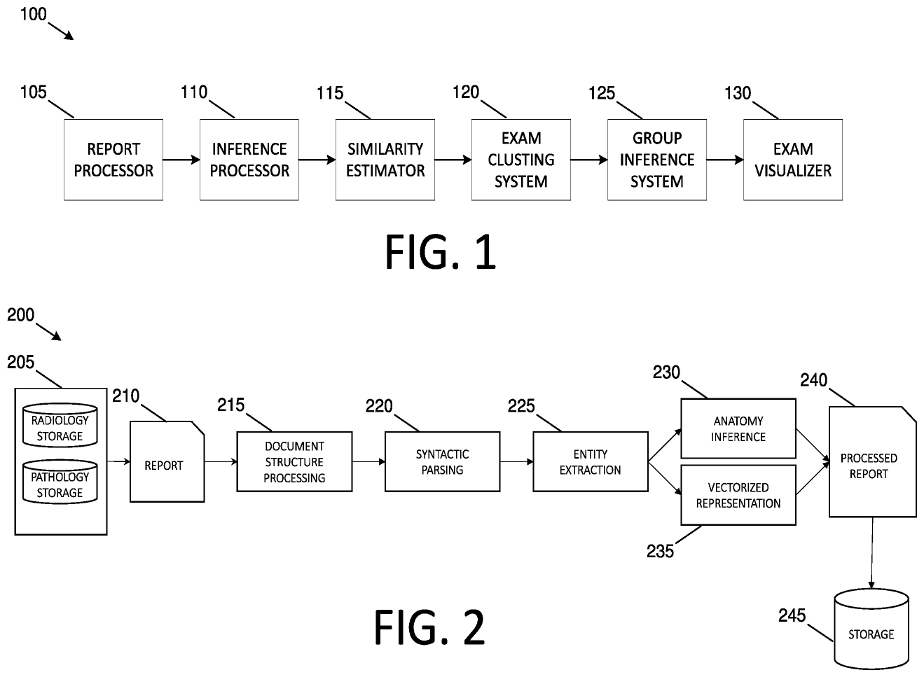

FIG. 1 illustrates an embodiment of a clustering system that groups patient radiology and pathology exams. The clustering system 100 classifies patient radiology and pathology exams according to the anatomy of the disease/pathology to enable gathering and presentation of relevant data and presentation in different disease contexts. While radiology and pathology reports are used as examples herein, other types of medical reports may be classified and grouped as well. The clustering system includes a report processor 105, an interference processor 110, a similarity estimator 115, an exam clustering system 120, a group inference system 124, and an exam visualizer 130.

The clustering system can link radiology and pathology reports, such as, for example, linking a pathology report regarding a breast biopsy with a corresponding X-ray of the breast. Further, this may also be done for prior reports, such as prior breast biopsy reports and prior breast X-rays, which may also be identified and linked to the current reports. The ability to link a pathology report to prior relevant radiology and X-ray reports, and the ability to link an X-ray to prior X-ray reports and pathology reports is one benefit and use of the clustering system.

The clustering system can analyze patient reports and data to estimate and determine similarities between reports for a given patient, such as, for example and without imputing limitation, similarities between a given pathology report and prior radiology reports. This may be accomplished by establishing similarities between the diagnosis section of pathology report with the diagnosis sections of the radiology reports. This is important because radiology and pathology reports are two different types of reports. Radiology reports, deal with the gross anatomies and are based on imaging, whereas pathology reports are based on based on a diagnosis at a cellular level. As a result, there may be few commonalities in terms of vocabulary between radiology and pathology reports, or the only commonality that exists may be between the diagnosis sections in pathology reports and impression sections in radiology reports, where anatomies, locations of diseases, and types of diseases are discussed.

In FIG. 1, the report processor 105 and inference processor 110 analyze the patient reports in order to provide data that is input into the similarity estimator 115. FIG. 2 illustrates a report processing pipeline 200 of the patient reports carried out by the report processor 105 and the inference processor 110. All Pathology and Radiology reports are stored in data storage 205 that includes radiology storage and pathology storage, but storage for other types of medical reports may be included as well. All the Pathology and Radiology reports 210 are first individually passed through the processing pipeline 200. The first step of the report processing pipeline 200 is the document structure processing 215 that identifies document structure such as sections, headers, paragraphs, sentences, etc. The document structure processing 215 receives a patient report 210 and analyzes the structure of the report. Because certain parts of the reports may have the most relevant information used to identify similarities, these parts of the reports may be identified. For example, as mentioned above, the diagnosis sections of pathology reports and impression sections of X-ray reports may be a good source to identify similarities. The document structure processing 215 may include a section typing model that determines different sections of the record and then determines the types of each of the sections identified. This may be done using trained sequence labeling models such Conditional Random Fields or rule-based models to identify section header and section content boundaries. In one example, these models may use vertical spacing and character attributes to segregate the document into sections, paragraphs and sentences. Both rule-based and CRF models that use vertical spacing and character attributes in the reports have been developed and shown to be robust and configurable. For example, at a specific medical site or system, a specific report structure is used. Knowledge of this report structure may be used to develop the section typing model. The output of the model will be sections of the document and an associated type of the section. The text in the sections may then be further processed to identify paragraphs and sentences. A vertical space and character attributes based sequence model is employed to identify paragraphs and sentences.

The document structure processing outputs are then fed into the syntactic parsing 220. The syntactic parsing 220 may include language models that process specific text in the document sections. For example, sentences may be syntactically parsed to identify noun phrases. The identified noun phrases are then processed by the entity extraction module 225 to, for example, identify anatomical regions (lower left lobe, gall bladder etc.), findings/diagnoses (pulmonary nodule, cirrhosis, hepatocellular carcinoma, etc.), and procedures (e.g., salpingostomy, colonoscopy etc.).

The anatomy inference module 230 then provides clinical ontology-based anatomy labels to the extracted entities (anatomies, findings/diagnoses, and procedures) based on exam meta data, document structure, and paragraph and sentence level information. The anatomy inference module is based on a dictionary of anatomies created using the Foundational Model of Anatomy (FMA) ontology in Unified Medical Language System Metathesaurus 2016AA version. All anatomical terms under FMA hierarchies of “Subdivision of cardinal body part” and ‘Organ’, which are commonly used in the real clinical setting, were included in the dictionary. This module provides mapping for phrases to their implied anatomical label e.g., pneumothorax is mapped to lung, salpingectomy to fallopian tube, and CT chest LLU to lung.

A vectorized representation module 235 provides vector representation of entities. Such representations may be produced by averaging the individual word vector representations provided by a language model trained on radiology and pathology reports (Word2Vec or GloVe). This training may be adapted to other types of medical reports as well, if other types of medical reports are used. Both simple averaging and weighted averaging methods may be employed. In the weighted averaging scheme the weights may be estimated from the Term Frequency Inverse Document Frequency (TF-IDF) values of each word in the phrase. Alternatively, embeddings generated from dynamic encoders such as BERT, Universal Sentence Encoder might also be used to produce vector representations of entities. In general multiple representations may be presented by the module.

The exam meta-data, document structure, the syntactic parse of the sentences, the extracted entities, their labels, are saved in a hierarchical data structure that preserves the relationships between the entities as a processed report 240. The following example data structures show how such data may be stored.

The following data structure may be output from the document structure processing 215:

```
{
  ID: Document ID (e.g., UG0000047)
  Text: Report Text
  Meta:{
    Procedure Type: Procedure type of the exam. (e.g. XR Chest 2
    Views)
    DTTMF: Date of exam report finalized
    Section: Field of view of exam (e.g. Chest)
  }
  Structure: [{ }, ... { }]
  Entities: [{ }, ... { }]
}
```

The syntactic parsing may take processed document in the above form and process it into a structure representation as follows:

```
[
  {
    ID: Structure Entity ID e.g., S0001
    Span: Character span of section text in the report Eg., (0,97)
    Type: Section
    Category: Impression
  },
  {
    ID: Structure Entity ID eg., P0001
    Span: Character span of paragraph text in the report E.g., (12,32)
    Type: Paragraph
  },
  {
    ID: Structure Entity ID e.g., s0001
    Span: Character span of sentence text in the report E.g., (12,48)
    Type: Sentence
  }
  ...
]
```

The entity extraction 255, anatomy inference 230, and vectorized representation 235 may process structure representation and produce an entity representation that becomes part of the processed report 240. An example of an entity representation is as follows:

```
{
  ID: Entity ID e.g., E0047
  Text: Actual Entity Text E.g. (Pneumothorax)
  Span: Character span of the text in the report E.g., (10,23)
  Type: Type of the entity. E.g. Anatomy, Finding, Symptom,
Measurement, Score, Diagnosis or Procedure
  Inferred Anatomy: Lung
  SENT_ID: Sentence to which this entity belongs to. E.g., SENT0017
  Vectors:[
    {
      value: [0.12, 0.23, 0.56, ...]
      source: ‘Averaging’
    },
    {
      value: [0.19, 0.19, 0.92, ...]
      source: ‘Weighted Average’
    },
    {
      value: [0.05, 0.11, 0.61, ...]
      source: ‘USE’
    },
    ...
}
```

After all of the reports are processed, the similarity estimator 115 estimates the similarities between each of the reports. For example, given a patient history of n exams, the similarity estimator 115 may compute a n² relevance matrix between the exams including n(n−1)/2 unique values. Each element in this matrix may contain a value in the range [0,1] (inclusive) indicating the relatedness of the two exams. In a supervised setting, these values may be estimated as relevance scores of a binary relatedness classifier (e.g., logistic regression, neural network, etc.). In an unsupervised setting, these values may be estimated as a relevance score of phrase-vectors which are computed using the noun phrases corresponding to entities extracted in the previous step. Any of these known techniques for determining the similarity between the reports may be used.

For example, the phrases found in a report are converted to a set of vectors. The vectors in the reports are compared by looking for similarities between the two sets of vectors. Based upon these vector comparisons, a similarity score may be determined using the various approaches discussed above. Note that, because a patient may have comorbidities, any report may be similar to a number of different reports.

Next, the exam clustering system 120 takes the similarity results and clusters the reports. This clustering may be done using various machine learning techniques. For example, in an unsupervised setting affinity propagation may be employed to identify clusters. In another embodiment, when two reports have a similarity score above specified threshold the reports may be linked to one another. In supervised setting a clustering may be achieved using a binary classifier that directly produces a relevance score between two reports. In another embodiment, clusters of reports may be determined directly via the anatomy labelling that comes out of the anatomy inference process based on identical or similar anatomies being examined in the reports. Any given report may be clustered/linked to a number of different reports because a patient may have multiple comorbidities. The exam clustering system 120 produces a set of related reports that are similar to one another.

Then the group inference system 125 may infer a group type from anatomy labeling and matching. Such labelling may come from standard dictionaries or databases describing medical conditions, terms, anatomy, etc., for example Systematized Nomenclature of Medicine SNOMED which is a standardized, vocabulary of clinical terminology that is used by physicians and other health care providers for the electronic exchange of clinical health information.

The labeled clusters then may be visualized for a medical professional by the exam visualizer 130. The exam visualizer 130 may include a processor and a display, where the processor processes the data related to the labelled clusters and provides that data to the display. The anatomy labels may be presented and the corresponding clusters related to them illustrated as time-ordered lists that demonstrate the evolution of patient through a particular disease or pathology in an anatomy. The medical professional may then select reports related to an anatomy of interest. For example, a pulmonologist might be interested in the evolution of an incidental lung nodule detected in a Breast cancer patient, while an orthopedist might be interested in tracking the progress of pelvic fracture. This can save the medical professional time by presenting all reports related to an anatomy of interest. In another use case, when the medical professional has a new report, the classifier system 100 may be used to determine all other related reports within the same cluster as the newly received report.

The clustering system 100 may be used, for example, in the following scenario. An elderly patient is being monitored regarding a recurrence of breast cancer. Accordingly, prior pathology and radiological reports related to the earlier bout of breast cancer are relevant to ongoing monitoring and diagnosis of the patient. The patient has had a number of other health issues and, as a result, has a number of other medical records. For example, the patient has rheumatoid arthritis and has had a number of X-ray regarding various joints as well as a hip replacement due to a fall and a knee replacement due to the rheumatoid arthritis. Further, the patient has had various lung infections that have resulted in a number of chest X-rays. There may also be other various types of X-rays and medical reports associated with the patient.

When the patient's doctor receives a new X-ray to evaluate the breast, the clustering system 100 may be used to quickly and accurately identify the other relevant reports for the doctor, such as the prior chest X-ray associated with the prior bout of cancer and the lung infections. An X-ray of breast or upper abdomen may also be included as it might include a relevant portion of the chest area. Also, prior pathology reports related to the prior bout of cancer will be identified. Further, X-rays related to the hip and knee replacements will be ignored as not relevant to the breast cancer. Other reports may be ignored as well because they are not relevant to the current diagnosis. As a result, the doctor does not have to waste time going through a large number of irrelevant reports. Also, if a large number of reports are present, a doctor may miss a relevant report in trying to get through all of them. The clustering system 100 will prevent this by determining the reports that are relevant to the current medical reports related to the diagnosis being sought by the doctor.

The clustering system helps to increase the ability of medical professionals to quickly and accurately find prior related medical exams Because of the automated processing done by the clustering system, the medical professional does not have to manually search through prior exams to find those of interest. When the number of prior exams is large, this search may be very time consuming or the medical professional may only do a cursory exam of the prior exam results. Further, using the various machine learning and data processing techniques of the clustering system, related reports may also be found with greater accuracy, especially in a time efficient manner as well as along bases of similarity that may not be practical when the reports are compared manually. For example, the clustering system may use a vectorized representation of the input data that allows the clustering system to identify similarity between examination results that a medical professional is unable to manually identify. These technological advancements solve the problem of the time and effort it takes to evaluate prior exams to determine which may be relevant to the current patient exam. This will allow a medical profession to more accurately diagnose a patient's condition as well as allowing for it to be done in a more efficient manner.

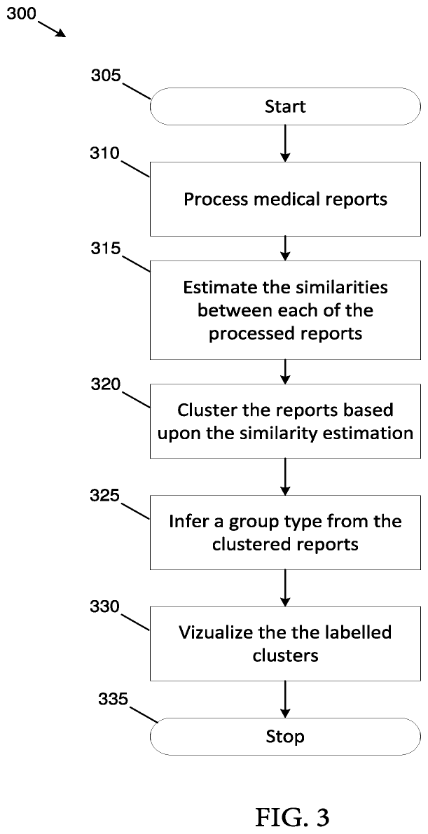

FIG. 3 illustrates a flowchart of a method performed by a clustering system that groups medical exams. The method 300 begins 305 and first receives and processes a group of medical reports 310. In the examples described above, the medical reports may be radiology and pathology reports, but may include other types of medical reports as well. The medical reports may be processed as further described in detail in FIG. 2. The processed medical reports are in a computerized format that allows for further processing. Next, the method 300 estimates similarities among the processed medical reports 315. This may be done by the similarity estimator as described above. The method 300 then clusters the reports based upon the similarity estimation 320. This may be done by the exam clustering system 120 as described above. Next, the method 300 may infer a group type from the clustered reports 325. The group inference system 125 described above may carry out this step of the method 300. Next, the method 300 visualizes the labeled clusters 330. The exam visualized 130 described above may carry out this step. The exam visualizer 130 may include a display and a processor configured to display the visualized data to a medical professional. The method 300 then ends 335.

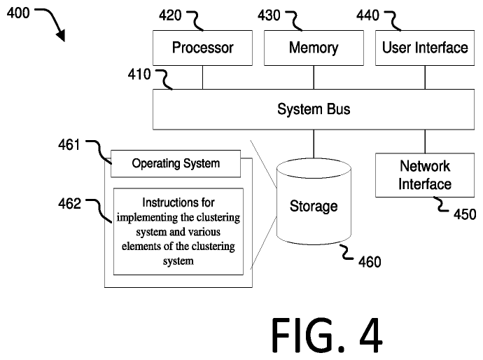

FIG. 4 illustrates an exemplary hardware device 400 for implementing the clustering system 100. This device may implement the whole clustering system 100 or may also implement various elements of it such as the report processor 105, inference processor 110, similarity estimator 115, exam clustering system 120, the group inference system 125, and the exam visualizer 130. As shown, the device 400 includes a processor 420, memory 430, user interface 440, network interface 450, and storage 460 interconnected via one or more system buses 410. It will be understood that FIG. 4 constitutes, in some respects, an abstraction and that the actual organization of the components of the device 400 may be more complex than illustrated.

The processor 420 may be any hardware device capable of executing instructions stored in memory 430 or storage 460 or otherwise processing data. As such, the processor may include a microprocessor, a graphics processing unit (GPU), field programmable gate array (FPGA), application-specific integrated circuit (ASIC), any processor capable of parallel computing, or other similar devices. The processor may also be a special processor that implements machine learning models.

The memory 430 may include various memories such as, for example L1, L2, or L3 cache or system memory. As such, the memory 430 may include static random-access memory (SRAM), dynamic RANI (DRAM), flash memory, read only memory (ROM), or other similar memory devices. The memory 430 may store intermediate results of the various elements of the clustering system 100 as they are passed from one element of the clustering system to another. An example would include the various data structures and data processed as described above.

The user interface 440 may include one or more devices for enabling communication with a user and may present information to users. For example, the user interface 440 may include a display, a touch interface, a mouse, and/or a keyboard for receiving user commands. In some embodiments, the user interface 440 may include a command line interface or graphical user interface that may be presented to a remote terminal via the network interface 450. The user interface 440 may be used to implement the exam visualizer 130 that presents the results of the grouping and labelling of the reports to the medical professional.

The network interface 450 may include one or more devices for enabling communication with other hardware devices. For example, the network interface 450 may include a network interface card (NIC) configured to communicate according to the Ethernet protocol or other communications protocols, including wireless protocols. Additionally, the network interface 450 may implement a TCP/IP stack for communication according to the TCP/IP protocols. Various alternative or additional hardware or configurations for the network interface 450 will be apparent.

The storage 460 may include one or more machine-readable storage media such as read-only memory (ROM), random-access memory (RAM), magnetic disk storage media, optical storage media, flash-memory devices, or similar storage media. In various embodiments, the storage 460 may store instructions for execution by the processor 420 or data upon which the processor 420 may operate. For example, the storage 460 may store a base operating system 461 for controlling various basic operations of the hardware 400. The storage 462 may store instructions for implementing the clustering system and various elements of the clustering system 100 such as the report processor 105, inference processor 110, similarity estimator 115, exam clustering system 120, the group inference system 125, and the exam visualizer 130. Further, the storage may store various data produced by the clustering system 100 such as the report processor 105, inference processor 110, similarity estimator 115, exam clustering system 120, the group inference system 125, and the exam visualizer 130, including the data structures described above.

It will be apparent that various information described as stored in the storage 460 may be additionally or alternatively stored in the memory 430. In this respect, the memory 430 may also be considered to constitute a “storage device” and the storage 460 may be considered a “memory.” Various other arrangements will be apparent. Further, the memory 430 and storage 460 may both be considered to be “non-transitory machine-readable media.” As used herein, the term “non-transitory” will be understood to exclude transitory signals but to include all forms of storage, including both volatile and non-volatile memories.

While the system 400 is shown as including one of each described component, the various components may be duplicated in various embodiments. For example, the processor 420 may include multiple microprocessors that are configured to independently execute the methods described herein or are configured to perform steps or subroutines of the methods described herein such that the multiple processors cooperate to achieve the functionality described herein. Such plurality of processors may be of the same or different types. Further, where the device 400 is implemented in a cloud computing system, the various hardware components may belong to separate physical systems. For example, the processor 420 may include a first processor in a first server and a second processor in a second server.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A method for classifying medical reports of a patient, comprising:
receiving a plurality of patient medical reports;
processing the plurality of patient medical reports to produce a processed report that extracts patient medical information;
estimating a relevance score between the plurality of medical reports based upon the extracted patient medical information and document vectors in processed medical report;
clustering similar patient medical reports using a trained classifier, wherein the trained classifier classifies patient medical reports based upon vectorized phrases corresponding to the extracted patient medical information;
inferring a group type for the clustered medical reports and labeling the clustered medical reports with the inferred group type using anatomy labeling matching based upon a standard dictionaries or databases including one of standard medical conditions, medical terms, and medical anatomies.

2. The method of claim 1, further comprising visualization of the labelled clustered medical reports on a display.

3. The method of claim 1, wherein processing the plurality of patient medical reports includes document structure processing.

4. The method of claim 3, wherein processing the plurality of patient medical reports includes syntactic parsing of an output of the document structure processing.

5. The method of claim 4, wherein processing the plurality of patient medical reports includes extracting entities from an output of the syntactic parsing using a vectorized representation of the extracted patient medical information.

6. The method of claim 5, wherein processing the plurality of patient medical reports includes determining an anatomy inference on the extracted entities.

7. The method of claim 1, further comprising:
receiving a current medical report for the patient;
processing the current medical report to produce a current processed report that extracts current patient medical information;
estimating the relevance score between the current medical report and the plurality of medical reports based upon the extracted current patient medical information and document vectors in the processed current medical report;
determining which of the clusters of medical reports the current report is similar to; and
labeling the current medical report with the inferred group type associated with the determined cluster of medical reports.

8. The method of claim 1, wherein the plurality of medical reports includes radiology reports and pathology reports.

9. The method of claim 1, wherein clustering similar medical reports includes using a machine learning model.

10. The method of claim 1, wherein clustering similar medical reports includes identifying medical reports that have a relevance score above a threshold value.

11. The method of claim 1, further comprising determining a current anatomy label for the current report based upon the processing of the current medical report wherein clustering similar medical reports includes using current anatomy label to match anatomy labels on the plurality of medical reports produced by the report processing.

12. The method of claim 1, wherein estimating the similarity between the plurality of medical reports is based upon one of anatomies identified in the reports, location of the disease identified in the reports, and the type of disease identified in the reports.

13. A device for classifying medical reports of a patient, comprising:
a memory;
a processor coupled to the memory, wherein the processor is further configured to:
receive a plurality of patient medical reports;
process the plurality of patient medical reports to produce a processed report that extracts patient medical information;
estimate the similarity between the plurality of medical reports based upon the extracted patient medical information;
cluster similar medical reports;
infer a group type for the clustered medical reports and labeling the clustered medical reports with the inferred group type; and
visualize the labelled clustered medical reports on a display.

14. The device of claim 13, wherein processing the plurality of patient medical reports includes document structure processing.

15. The device of claim 13, wherein processing the plurality of patient medical reports includes syntactic parsing of an output of the document structure processing.

16. The device of claim 15, wherein processing the plurality of patient medical reports includes extracting entities from an output of the syntactic parsing.

17. The device of claim 16, wherein processing the plurality of patient medical reports includes determining an anatomy inference on the extracted entities.

18. The device of claim 13, wherein the processor is further configured to:
receive a current medical report for the patient;
process the current medical report to produce a current processed report that extracts patient medical information;
estimate the similarity between the current medical report and the plurality of medical reports;
determine which of the clusters of medical reports the current report is similar to;
label the current medical report with the inferred group type associated with the determined cluster of medical reports; and
visualize the current medical report on a display.

19. The device of claim 13, wherein the plurality of medical reports include radiology reports and pathology reports.

20. The device of claim 13, wherein clustering similar medical reports includes using a machine learning model.

21. The device of claim 12, wherein clustering similar medical reports includes identifying medical reports that have a similarity score above a threshold value.

22. The device of claim 12, wherein the processor is further configured to determine a current anatomy label for the current report based upon the processing of the current medical report wherein clustering similar medical reports includes using current anatomy label to match anatomy labels on the plurality of medical reports produced by the report processing.

23. The device of claim 12, wherein estimating the similarity between the plurality of medical reports is based upon one of anatomies identified in the reports, location of the disease identified in the reports, and the type of disease identified in the reports.
